(19) United States
(12) Patent Application Publication
(10) Pub. No.: US 2025/0066314 A1
(43) Pub. Date: Feb. 27, 2025

(54) PROCESS OF COUPLING ALDEHYDES

(71) Applicant: UOP LLC, Des Plaines, IL (US)

(72) Inventors: Erin M. Broderick, Arlington Heights, IL (US); Amanda Hickman, Evanston, IL (US); Gregory B. Kuzmanich, Arlington Heights, IL (US)

(21) Appl. No.: 18/453,220

(22) Filed: Aug. 21, 2023

(51) Int. Cl. C07D 307/48; B01J 31/22

(52) U.S. Cl. CPC C07D 307/48; B01J 31/2273; B01J 2231/42

(57) ABSTRACT

A process of coupling aldehydes is disclosed. The process comprises passing an aldehyde stream and a catalyst stream comprising a homogeneous catalyst in an alcohol solution to a reactor. The aldehyde stream is mixed with the homogeneous catalyst in the alcohol solution. A furoin based molecule is precipitated from the solution in the reactor. A liquid stream comprising the homogeneous catalyst in the alcohol solution is recovered from the reactor. The liquid stream comprising the homogeneous catalyst in the alcohol solution is recycled to the reactor to couple the fresh supply of the aldehyde stream.

### FIELD

The field is related to a process of coupling aldehydes. The field may particularly relate to a process of coupling aldehydes and recycling a catalyst stream for coupling aldehydes.

### BACKGROUND

As the demand for fuel increases worldwide, there is increasing interest in producing fuels and blending components from sources other than crude oil. Often referred to as biorenewable sources, these sources include, but are not limited to, plant oils such as corn, rapeseed, canola, soybean, microbial oils such as algal oils, animal fats such as inedible tallow, fish oils and various waste streams such as yellow and brown greases and sewage sludge. A common feature of these sources is that they are composed of glycerides and free fatty acids (FFA). Both triglycerides and the FFAs contain aliphatic carbon chains having from about 8 to about 24 carbon atoms. The aliphatic carbon chains in triglycerides or FFAs can be fully saturated or mono, di or poly-unsaturated.

With an increased focus on sustainability, the need for sustainable aviation fuels (SAF) has grown due to the decrease in greenhouse gases (GHG) emissions of SAF compared to petrochemically sourced fuel. There is increasing support and demand across the globe for SAF with the government offering subsidies and mandating the production of carbon-neutral jet fuel. In recent years, considerable research has been devoted to finding effective and efficient means of producing the SAF. As the demand for SAF increases worldwide, there is an increasing interest in sources other than petroleum crude oil for producing the SAF.

Light weight olefins have traditionally been produced through the process of steam or catalytic cracking. Light weight olefins serve as feeds for the production of numerous chemicals. However, the use of petroleum sources as feed leads to GHG emissions. Researchers and refineries are looking for alternate sources for these processes. The search for alternative materials for light weight olefin production has led to the use of oxygenates. Oxygenates may provide non-petroleum-based routes for the production of olefins and other hydrocarbons. However, there is a need for a consistent supply of sources for oxygenates to meet the demand of lightweight olefins.

Azolium compounds such as a thiazolium salt, an imidazolium salt and a triazolium salt are known to be used as catalysts for aldehyde coupling reactions such as benzoin condensation. The coupled product of aldehyde coupling reactions include alkanes which can be converted to other products including fuels. However, these azolium compounds are difficult to recover and, hence, their industrial use as catalysts is difficult. For this reason, azolium compounds are used as supported catalysts on organic or inorganic carriers. However, these azolium compound supported catalysts have problems such as insufficient yield, precipitous reduction in catalyst activity, and poor recyclability.

The anticipation of stagnation in growth of high fructose corn syrup could lead to stranded sugars in the future. It would be desirable to have an effective pathway for converting sugars to hydroxymethfurfural (HMF) which may be processed to produce sustainable aviation fuel meeting fuel specifications and to other chemicals such as olefins.

### SUMMARY OF THE INVENTION

A process of coupling aldehydes is disclosed. The process discloses contacting an aldehyde containing stream with a catalyst stream of a catalyst in an alcohol solution. The coupling reaction produces a furoin based molecule which is withdrawn. A liquid stream of catalyst in the alcohol solution is also withdrawn from the reactor. The process recycles the used catalyst back to the reactor to couple a fresh supply feed of aldehyde. It is demonstrated that the recycled catalyst for the coupling reaction has produced a commensurate yield compared to fresh catalyst. Also, the furoin based molecule may be processed to produce SAF or olefin.

### BRIEF DESCRIPTION OF THE DRAWINGS

FIG. 1 is a simplified process flow diagram of the process of coupling aldehydes in accordance with an exemplary embodiment of the present disclosure.

FIG. 2 is a graph plotted between the 5,5′-di(hydroxymethyl)furoin (DHMF) % yield vs time (min) in accordance with another exemplary embodiment of the present disclosure.

FIG. 3 is a graph plotted between the variation of DHMF % yield vs water content of a used solvent in accordance with yet another exemplary embodiment of the present disclosure.

FIG. 4 is a graph plotted to show the variation of DHMF % yield with a mole ratio of the aldehyde and catalyst at different water concentrations in alcohol as the solvent in accordance with still another exemplary embodiment of the present disclosure.

FIG. 5 is a graph plotted to show the variation of DHMF % yield with a mole ratio of the aldehyde and catalyst for different catalysts in accordance with yet another exemplary embodiment of the present disclosure.

FIG. 6 is a graph plotted to show the variation of DHMF % yield for fresh and a recycled catalyst in accordance with still another exemplary embodiment of the present disclosure.

FIG. 7 is a graph plotted to show the variation of C10% yield with time for hydrodeoxygenation (HDO) of furoin in accordance with yet another exemplary embodiment of the present disclosure.

FIG. 8 is a graph plotted to show the variation of C10% yield with hydrogen pressure in accordance with still another exemplary embodiment of the present disclosure.

FIG. 9 is a graph plotted to show the variation of C10% yield with furoin:catalyst weight ratio in accordance with yet another exemplary embodiment of the present disclosure.

FIG. 10 is a graph plotted to show the variation of C10% yield with temperature in accordance with still another exemplary embodiment of the present disclosure.

### DEFINITIONS

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take a main product from the bottom.

As used herein, the term “a component-rich stream” means that the rich stream coming out of a vessel has a greater concentration of the component than the feed to the vessel.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T10” means the temperature at which 10 mass percent of the sample boils using ASTM D-86 or TBP.

As used herein, the term “initial boiling point” (IBP) means the temperature at which the sample begins to boil using ASTM D2887, ASTM D-86 or TBP, as the case may be.

As used herein, the term “end point” (EP) means the temperature at which the sample has all boiled off using ASTM D2887, ASTM D-86 or TBP, as the case may be.

As used herein, the term “jet fuel range material” means hydrocarbons boiling in the range of an IBP between about 85° C. (185° F.) and about 135° C. (275° F.) or a T5 between about 110° C. (230° F.) and about 160° C. (320° F.) and the “recycle cut point” comprising a T95 between about 295° C. (563° F.) and about 315° C. (599° F.) using the TBP distillation method. Hydrocarbons beyond the “recycle cut point”

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “Cₓ” is to be understood to refer to molecules having the number of carbon atoms represented by the subscript “x”. Similarly, the term “Cₓ₋” refers to molecules that contain less than or equal to x and preferably x and less carbon atoms. The term “Cₓ+” refers to molecules with more than or equal to x and preferably x and more carbon atoms.

As used herein, the term “carbon number” refers to the number of carbon atoms per hydrocarbon molecule and typically a paraffin molecule.

As used herein, the term “alcohol” refers to an at least mono-hydroxy-substituted alkane. A typical alcohol comprises a (C₁-C₁₂) alkyl moiety substituted at a hydrogen atom with one or more hydroxyl groups. Alcohol includes methanol, ethanol, n-propanol, i-propanol, n-butanol, i-butanol, s-butanol, t-butanol, n-pentanol, i-pentanol, hexanol, cyclohexanol, heptanol, octanol, nonanol, decanol, and the like. The carbon atom chain in alcohols can be straight, branched or cyclic. Alcohol can be mono-hydroxy, di-hydroxy, tri-hydroxy, and the like.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” means jet fuel comprising hydrocarbons not sourced from fossil fuels.

As used herein, the abbreviation “LHSV” means liquid hourly space velocity, which is defined as the volumetric flow rate of liquid per hour divided by the catalyst volume, where the liquid volume and the catalyst volume are in the same volumetric units.

As used herein, the term “predominant” or “predominate” means greater than 50%, suitably greater than 75% and preferably greater than 90%.

### DETAILED DESCRIPTION

With an increased focus on sustainability, the need for SAF has grown due to the decrease in GHG emissions of SAF compared to petrochemically sourced fuel. In addition, the stagnation of growth in high fructose corn syrup could lead to stranded sugars in the future. In the present disclosure, a process for coupling aldehydes including converting sugars via HMF to SAF and olefins is demonstrated for chemical and technoeconomic feasibility.

The disclosure includes a novel process for coupling aldehydes. The disclosed process is unique because it demonstrates a novel reuse process for the homogeneous catalyst used in the benzoin condensation to produce an acyloin. Examples of successfully reusing the homogeneous catalysts are not found in the prior art. Recycling the homogeneous catalyst is vital to achieving an economically viable process. Since the present process discloses that the homogeneous catalyst can be reused, the catalyst will be cost effective as opposed to a consumable.

The present disclosure provides a process of coupling aldehydes of an aldehyde containing stream. The aldehydes of the process may be specifically termed, furaldehydes. The aldehyde stream may be obtained from biological resources or biomass. In accordance with the present disclosure, the aldehyde stream may be obtained from lignocellulosic biomass such as corn stover, bagasse, wheat straw, rice straw, and wood.

The process discloses coupling of aldehydes obtained from a biomass to provide C₁₀-C₂₂ alkanes. The disclosed process includes using renewable biomass resources to diminish the reliance on petroleum-based liquid fuels to produce C₁₀-C₂₂ alkanes which may be processed to produce jet fuel meeting SAF requirements or converted to desired olefins.

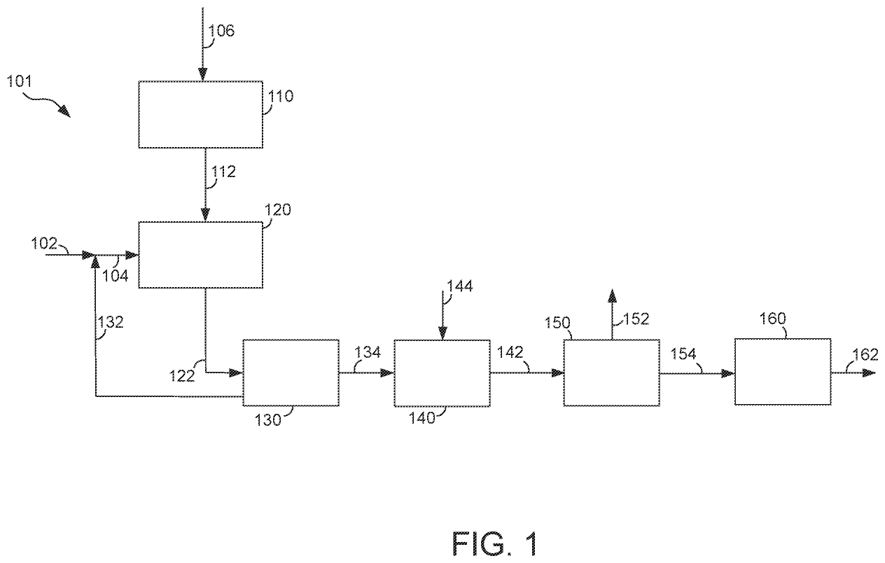

As shown in FIG. 1, the process of coupling aldehydes 101 comprises a reactor 120 for coupling aldehydes, a HDO reactor 140, and a processing unit 160. An aldehyde containing stream 112 and a catalyst stream comprising a catalyst in an alcohol solution 102 are passed to the reactor 120. The alcohol solution of the catalyst stream 102 may comprise one or more alcohols selected from methanol, ethanol, propanol, butanol, ethanolamine, phenol, and cresol. Further, the alcohol solution of the catalyst stream 102 may comprise one or more alcohols from C₁-C₂₀ alcohol and aromatic alcohol. In accordance with an exemplary embodiment of the present disclosure, the reactor 120 is a benzoin condensation reactor. A recycle stream 132, as described hereinafter in detail, is also passed to the reactor 120.

In an aspect, the catalyst in the catalyst stream 102 is a homogeneous catalyst. The catalyst may comprise a N-heterocyclic carbene catalyst. In an exemplary embodiment, the catalyst is selected from 1,3,4-triphenyl-4,5-dihydro-1H-1,2,4-triazol-5-ylidene (TPT), 1,3-di-mesityl-butyl-imidazolin-2-ylidene and 1,3-dialkylimidazolin-2-ylidene.

The aldehyde containing stream 112 may be taken from any suitable source 110. In an exemplary embodiment, the source of the aldehyde containing stream 112 is a dehydration unit 110. The aldehyde containing stream 112 may comprise at least two aldehydes that may be different from each other. In an aspect of the present disclosure, a biomass derived carbohydrate stream in line 106 may be dehydrated in the dehydration unit in the aldehyde source 110 to provide the aldehyde containing stream 112. In an embodiment, the biomass derived carbohydrate stream in line 106 may comprise C₅-C₆ sugars. In another embodiment, the biomass derived carbohydrate stream in line 106 may comprise one or more of cellulose, fructose, and glucose.

In an aspect of the present disclosure, the aldehyde containing stream in line 112 comprises one or more aldehydes having formula (I):

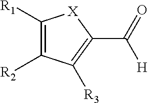

wherein R1, R2, and R3 is selected from a group comprising hydrogen, C₁-C₂₀ alkyl groups, aromatic groups, heteroatoms, and oxygenated groups comprising alcohols, ethers, carbonyls, esters, and hydroxyls, and X is selected from a group comprising heteroatoms, oxygen, sulfur, and nitrogen. All of the R₁, R₂, and R₃ in the formula (I) may be the same. Further, any two of the R₁, R₂, and R₃ in the formula (I) may be the same. Furthermore, R₁, R₂, and R₃ in the formula (I) may each be different from each other.

In an embodiment, the aldehyde containing stream in line 112 comprises furfural. In an exemplary embodiment, the aldehyde containing stream in line 112 may comprise 2-hydroxymethylfurfural (HMF).

In accordance with an exemplary embodiment of the present disclosure, a recycled liquid stream in line 132 is combined with the catalyst stream in line 102 to provide a combined feed stream in line 104 which is passed to the reactor 120. In an alternate embodiment, the catalyst stream in line 102 and the recycled liquid stream in line 132 may be passed separately to the reactor 120.

In the reactor 120, the aldehyde containing stream in line 112 is mixed with the catalyst in the alcohol solution, so the aldehyde coupling reaction takes place. The aldehyde coupling reaction is a coupling reaction between two aldehyde molecules. The aldehyde coupling reaction can also be referred to as a “coupling reaction”. The aldehyde coupling reaction may include self-condensation (self-coupling) as well as cross-condensation (cross-coupling). A coupling reaction between two aromatic aldehydes (such as benzaldehyde) is generally called a “benzoin condensation”. The aldehyde coupling reaction in the present disclosure, however, is not limited to the benzoin condensation.

After mixing, a furoin based molecule is formed which is precipitated out of the mixture within the reactor 120. The precipitation of the furoin based molecule takes place within the reactor 120 autogenously, in the absence of adding any solvent or agent for precipitation. Also, the precipitation of the furoin based molecule takes place within the reactor 120, so another vessel for precipitation is not required in accordance with the present process 101. After the coupling reaction is complete, a coupling reaction effluent stream is withdrawn in line 122 from the reactor 120. The coupling reaction effluent stream in line 122 may be passed to a separator 130 to separate the precipitated furoin based molecule. The furoin based molecule is collected and withdrawn from the separator 130 in a coupling reaction product stream in line 134. In an exemplary embodiment, the furoin based molecule may be collected via a centrifuge 130 to remove liquid from the furoin molecules. After the collection and separation of the furoin based molecule in the separator 130, a liquid stream remains. The liquid stream comprises the catalyst in the alcohol solution. The catalyst in this leftover liquid stream may be referred to as a spent catalyst after the coupling reaction in the reactor 120. The liquid stream comprising the catalyst in the alcohol solution is recovered and withdrawn from the separator 130 in line 132.

In accordance with the present disclosure, the liquid stream comprising the catalyst in the alcohol solution in line 132 may be recycled directly to the reactor 120 to continuously catalyze coupling the freshly supplied aldehyde containing stream in line 112. The disclosed process of coupling aldehydes is unique compared to a typical or conventional processes. Successful recycling or reusing a spent catalyst after the coupling reaction is hereby demonstrated unlike conventional processes. The conventional processes usually resort to separation and further treatment of catalyst which may be used further. Also, the successful use of the recycled catalyst on the coupling reaction is hereby demonstrated. We have found that after the coupling reaction, the catalyst stream can be successfully recycled to couple the aldehyde containing stream in the reactor and produce a commensurate yield of furoin based molecule when compared with the coupling reaction in the presence of a fresh, unrecycled catalyst. Recycling of the catalyst is vital to achieving an economically viable process. We have disclosed an economical process for the production of furoin based molecule which in turn produces valuable alkanes from a biomass derived aldehyde stream. The alkanes may be processed to produce SAF or olefins.

Returning back to the reactor 120, the liquid stream comprising the catalyst in the alcohol solution in line 132 is recycled back to the reactor 120 along with a fresh supply of the catalyst supplied in an alcohol solution in line 102 via line 104. The precipitated furoin based molecule in the coupling reaction product stream in line 134 is withdrawn from the separator 130 for further processing to produce alkanes. In accordance with the present disclosure, the furoin based molecule in the coupling reaction product stream in line 134 may comprise one or more compounds having formula (II):

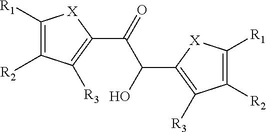

wherein R₁, R₂, and R₃ is selected from a group comprising hydrogen, C₁-C₂₀ alkyl groups, aromatic groups, heteroatoms, and oxygenated groups comprising alcohols, ethers, carbonyls, esters, and hydroxlys, and X is selected from a group comprising heteroatoms, oxygen, sulfur, and nitrogen. All of the R₁, R₂, and R₃ in the formula (I) may be same. Further, any two of the R₁, R₂, and R₃ in the formula (I) may be the same. Furthermore, R₁, R₂, and R₃ in the formula (I) may be different from each other. In an exemplary embodiment, the furoin based molecule in the coupling reaction product stream in line 134 may comprise one or both of di(hydroxymethyl)furoin (DHMF), specifically 5,5′-di(hydroxymethyl) furoin and furoin.

The furoin based molecule in the coupling reaction product stream in line 134 is converted to valuable alkanes by hydrodeoxygenation of the furoin based molecule. The total number of carbons in the furoin based molecule may be equal to the sum of the two aldehydes employed for the coupling reaction. The furoin based molecule in the coupling reaction product stream in line 134 is passed to a HDO reactor 140 for hydrodeoxygenation of the coupling products in the presence of a HDO catalyst. The HDO catalyst in the HDO reactor 140 may also be referred to as a hydrotreating catalyst. The furoin based molecule in the coupling reaction product stream in line 134 may be subjected to hydrotreating in the HDO reactor 140. A hydrogen gas stream in line 144 is also passed to the HDO reactor 140.

Hydrotreating may also include hydrocracking of the feed in the HDO reactor 140. The hydrotreating reactions remove contaminants from the feed and product streams, while the hydrocracking reactions create usable lighter weight products. The primary hydrotreating reactions may include sulfur and nitrogen removal as well as olefin saturation. The products of these reactions are the corresponding contaminant-free hydrocarbon, along with H2S and NH3. Other treating reactions may include oxygen, metals and halide removal, and aromatic saturation. The reactions are typically carried out at elevated pressures and temperatures in a hydrogen atmosphere.

The HDO reactor 140 may comprise one or more HDO catalyst beds and include one or more reactor vessels. HDO catalyst in the HDO reactor 140 may include any of those well known in the art such as nickel or nickel/molybdenum dispersed on a high surface area support. Other catalysts include one or more noble metals dispersed on a high surface area support. Non-limiting examples of noble metals include platinum and/or palladium dispersed on gamma-alumina. Reaction conditions in the HDO reactor 140 may include a relatively low pressure of about 3447 kPa (500 psia) to about 6895 kPa (1000 psia), a temperature of about 200° C. (392° F.) to about 400° C. (752° F.), and a LHSV of about 0.5 hr⁻¹ to about 10 hr⁻¹. In another embodiment, the reaction conditions in the HDO reactor 140 may include the same relatively low pressure of about 3447 kPa (500 psia) to about 6895 kPa (1000 psia), a temperature of about 288° C. (550° F.) to about 345° C. (653° F.) and a liquid hourly space velocity of about 1 hr⁻¹ to about 4 hr^{-1.}

In the HDO reactor 140, the furoin based molecule in the coupling reaction product stream in line 134 is deoxygenated to form water and a hydrocarbon. The reaction product from the deoxygenation reactions comprises both a deoxygenated liquid and a gaseous component. A HDO reaction product stream in line 142 is withdrawn and passed to a HDO separation section 150 where a deoxygenated liquid stream in line 154 is separated from a gaseous stream in line 152. The deoxygenated liquid stream in line 154 comprises a hydrocarbon fraction which comprises primarily paraffins perhaps having a predominant concentration of paraffins in the range of about 10 to about 18 carbon atoms. The gaseous stream in line 152 comprises hydrogen, carbon dioxide, carbon monoxide, water vapor, propane and light hydrocarbons. The deoxygenated liquid stream in line 154 may also be referred to as a HDO reactor effluent stream in line 154. In an exemplary embodiment, the deoxygenated liquid stream in line 154 may comprise C₁₂ to C₂₂ alkanes. In another exemplary embodiment, the deoxygenated liquid stream in line 154 may comprise C₁₀ to C₁₂ alkanes. The deoxygenated liquid stream in line 154 may be processed in the processing unit 160 to convert the alkanes into a desired product.

In an aspect, the deoxygenated liquid stream in line 154 may be processed to produce liquid jet fuel (SAF). In an exemplary embodiment, the processing unit 160 is a hydroisomerization unit 160. Hydroisomerization is employed to improve cold flow properties of product jet fuel. Hydroisomerization or hydrodewaxing is a hydroprocessing process that increases the alkyl branching on a hydrocarbon backbone in the presence of hydrogen and hydroisomerization catalyst to improve cold flow properties of the hydrocarbon.

In the hydroisomerization unit 160, the deoxygenated liquid stream in line 154 is contacted with a hydroisomerization catalyst in the hydroisomerization reactor under hydroisomerization conditions to hydroisomerize the normal paraffins to branched paraffins. The hydroisomerization unit 160 may include one or more reactors, a stripper column, and fractionation column.

Hydroisomerization, including hydrodewaxing, of the normal hydrocarbons in the hydroisomerization reactor can be accomplished over one or more beds of hydroisomerization catalyst, and the hydroisomerization reactor 160 may be operated in a co-current mode of operation. Fixed bed, trickle bed down-flow or fixed bed liquid filled up-flow modes are both suitable.

Suitable hydroisomerization catalysts may comprise a metal of Group VIII (IUPAC 8-10) of the Periodic Table and a support material. Suitable Group VIII metals include platinum and palladium, each of which may be used alone or in combination. The hydroisomerization catalyst may include non-noble metals which are not as susceptible to sulfur deactivation in a sour environment. Examples of suitable non-noble metals include nickel (Ni), molybdenum (Mo), cobalt (Co), tungsten (W), manganese (Mn), copper (Cu), zinc (Zn), or ruthenium (Ru). Mixtures of hydrogenation metals may also be used such as Co/Mo, Ni/Mo and Ni/W. The amount of hydrogenation metal or metals may range from 0.1 to 5 wt. %, based on the catalyst weight. Methods of loading metal onto the support material include, for example, impregnation of the support material with a metal salt of the hydrogenation component and heating. The catalyst support material containing the hydrogenation metal may also be sulfided prior to use.

The support material may be amorphous or crystalline. Suitable support materials include amorphous alumina, amorphous silica-alumina, ferrierite, ALPO-31, SAPO-11, SAPO-31, SAPO-37, SAPO-41, SM-3, MgAPSO-31, FU-9, NU-10, NU-23, ZSM-12, ZSM-22, ZSM-23, ZSM-35, ZSM-48, ZSM-50, ZSM-57, MeAPO-11, MeAPO-31, MeAPO-41, MgAPSO-11, MgAPSO-31, MgAPSO-41, MgAPSO-46, ELAPO-11, ELAPO-31, ELAPO-41, ELAPSO-11, ELAPSO-31, ELAPSO-41, laumontite, cancrinite, offretite, hydrogen form of stillbite, magnesium or calcium form of mordenite, and magnesium or calcium form of partheite, each of which may be used alone or in combination. ALPO-31 is described in U.S. Pat. No. 4,310,440. SAPO-11, SAPO-31, SAPO-37, and SAPO-41 are described in U.S. Pat. No. 4,440,871. SM-3 is described in U.S. Pat. Nos. 4,943,424; 5,087,347; 5,158,665; and 5,208,005. MgAPSO is a MeAPSO, which is an acronym for a metal aluminumsilicophosphate molecular sieve, where the metal, Me, is magnesium (Mg). Suitable MgAPSO-31 catalysts include MgAPSO-31. MeAPSOs are described in U.S. Pat. No. 4,793,984, and MgAPSOs are described in U.S. Pat. No. 4,758,419. MgAPSO-31 is a preferred MgAPSO, where 31 means a MgAPSO having structure type 31. Many natural zeolites, such as ferrierite, that have an initially reduced pore size can be converted to forms suitable for olefin skeletal isomerization by removing associated alkali metal or alkaline earth metal by ammonium ion exchange and calcination to produce the substantially hydrogen form, as taught in U.S. Pat. Nos. 4,795,623 and 4,924,027. Further catalysts and conditions for skeletal isomerization are disclosed in U.S. Pat. Nos. 5,510,306, 5,082,956, and 5,741,759. The hydroisomerization catalyst may also comprise a modifier selected from the group consisting of lanthanum, cerium, praseodymium, neodymium, samarium, gadolinium, terbium, and mixtures thereof, as described in U.S. Pat. Nos. 5,716,897 and 5,851,949. Other suitable support materials include ZSM-22, ZSM-23, and ZSM-35, which are described for use in dewaxing in U.S. Pat. No. 5,246,566 and in the article entitled S. J. Miller, “New Molecular Sieve Process for Lube Dewaxing by Wax Isomerization,” 2 Microporous Materials 439-449 (1994). U.S. Pat. Nos. 5,444,032 and 5,608,968 teach a suitable bifunctional catalyst which is constituted by an amorphous silica-alumina gel and one or more metals belonging to Group VIIIA and is effective in the hydroisomerization of long-chain normal paraffins containing more than 15 carbon atoms. U.S. Pat. Nos. 5,981,419 and 5,908,134 teach a suitable bifunctional catalyst which comprises: (a) a porous crystalline material isostructural with beta-zeolite selected from boro-silicate (BOR-B) and boro-alumino-silicate (Al-BOR-B) in which the molar SiO₂:Al₂O₃ ratio is higher than 300:1; (b) one or more metal(s) belonging to Group VIIIA, selected from platinum and palladium, in an amount comprised within the range of from 0.05 to 5% by weight. V. Calemma et al., App. Catal. A: Gen., 190 (2000), 207 teaches yet another suitable catalyst. Alumina or silica may be added to the support material.

In an exemplary embodiment, DI-200 available from UOP LLC in Des Plaines, Illinois may be a suitable hydroisomerization catalyst.

Hydroisomerization conditions generally include a temperature of about 150° C. (302° F.) to about 450° C. (842° F.) and a pressure of about 1724 kPa (abs) (250 psia) to about 13.8 MPa (abs) (2000 psia). In another embodiment, the hydroisomerization conditions include a temperature of about 300° C. (572° F.) to about 360° C. (680° F.) and a pressure of about 3102 kPa (abs) (450 psia) to about 6895 kPa (abs) (1000 psia).

A hydroisomerized stream from the hydroisomerization reactor is a branched-paraffin-rich stream. By the term “branched-paraffin-rich” it is meant that the effluent stream has a greater concentration of branched paraffins than the stream entering the hydroisomerization reactor, and preferably comprises greater than 50 mass-% branched paraffins of the total paraffin content. It is envisioned that the hydroisomerized stream may contain 80, 90 or 95 mass-% branched paraffins of the total paraffin content. The optimal amount of remaining normal paraffins may depend on the selectivity of the hydroisomerization catalysts but might typically be between 1-7 wt-%.

The hydroisomerized stream may be passed to a hydroisomerization stripper column to separate a hydroisomerized liquid stream from a hydroisomerized vapor stream. The hydroisomerized vapor stream comprising light gases is removed. The hydroisomerized liquid stream is passed to a product distillation column to fractionate the hydroisomerized liquid stream and provide a jet fuel stream. The jet fuel stream so produced has a T5 of about 115° C. (239° F.) to about 130° C. (266° F.) and a T90 of about 240° C. (464° F.) to about 270° C. (518° F.). We have observed that the jet fuel stream so produced meets the ASTM D7566 jet fuel specification. The produced jet fuel stream may be withdrawn from the unit 160 in line 162.

In accordance with another embodiment of the present disclosure, the deoxygenated liquid stream in line 154 may be processed to produce olefins for linear alkylbenzene benzene (LAB) production. In an alternate embodiment, the processing unit 160 is a dehydrogenation unit 160. The dehydrogenation unit 160 may include one or more reactors, columns, sorptive separation zones, and extraction zone.

Alkylbenzenes (phenyl-alkanes) are prepared by the alkylation of benzenes. Alkylbenzenes have found many utilities, the most prominent of which is to make alkylbenzene sulfonates for use in laundry detergents and similar products. The performance of the alkylbenzene sulfonate in a detergent composition will be affected by the nature of the alkyl group, for instance, its length and configuration, especially branching.

Linear alkylbenzene sulfonates (LABS) are manufactured from linear alkylbenzenes (LAB). A typical process for producing LAB consists of dehydrogenating linear paraffins to linear olefins and then alkylating benzene with the linear olefins in the presence of a catalyst such as hydrofluoric acid or a solid, acidic catalyst.

Any suitable catalyst may be used for the dehydrogenation process. Many types of dehydrogenation catalysts are known as exemplified by U.S. Pat. Nos. 3,274,287; 3,315,007; 3,315,008; 3,745,112; and 4,430,517. Often the dehydrogenation catalysts are platinum group metal containing catalysts. One preferred catalyst is a layered composition comprising an inner core and an outer layer bonded to the inner core, where the outer layer comprises a refractory inorganic oxide having uniformly dispersed thereon at least one platinum group (Groups 8-10 of the periodic table) metal and at least one promoter metal, and where at least one modifier metal is dispersed on the catalyst composition.

The dehydrogenation may be conducted in the liquid phase or in a mixed vapor-liquid phase, but preferably in the vapor phase. Typical dehydrogenation conditions involve a temperature of from about 400° C. (752° F.) to about 900° C. (1652° F.) and preferably from about 420° C. (788° F.) to about 550° C. (1022° F.). Generally, for normal paraffins, the lower the molecular mass the higher the temperature required for comparable conversion. Pressures are generally from about 1 kPa(g) (0.15 psi(g)) to about 1000 kPa(g) (145 psi(g)), preferably between about 100 kPa(g) (14.5 psi(g)) and 400 kPa(g) (58.0 psi(g)), and a LHSV of from about 0.1 to about 100 hr^{-1.}

The deoxygenated liquid stream in line 154 is passed to a dehydrogenation section to convert the paraffins in the deoxygenated liquid stream to an olefin-containing stream. In an exemplary embodiment, the olefin containing stream from the dehydrogenation section comprises olefins having from 8 to 16 carbons. The olefin-containing stream is then passed to a selective hydrogenation section to convert diolefins to mono-olefins. Hydrogen is supplied to the selective hydrogenation section. The olefin-containing stream is then passed from selective hydrogenation section to a sorbent separation zone to remove aromatic by-products by sorption and provide a sorption effluent stream which is passed to a downstream alkylation reactor.

The sorptive separation zone may include fixed bed or moving or fluidized sorbent bed systems, but the fixed bed system is preferred. The sorbent may be installed in one or more vessels and in either series or parallel flow. The flow of the feedstock containing the aromatic by-products through the sorptive separation zone is preferably performed in a parallel manner so that one or more sorption beds can undergo regeneration while one or more beds are removing aromatic by-products.

Suitable sorbents may be selected from materials which exhibit the primary requirement of selectivity for the aromatic by-products, and which are otherwise convenient to use. Suitable sorbents include, for example, molecular sieves, silica, activated carbon activated charcoal, activated alumina, silica-alumina, clay, cellulose acetate, synthetic magnesium silicate, macroporous magnesium silicate, and/or macroporous polystyrene gel.

Alkylbenzenes (phenyl-alkanes) are prepared by the alkylation of benzenes in the alkylation reactor. In the alkylation reactor, the sorption effluent stream having a reduced concentration of aromatic by-products is mixed with a benzene stream. The sorption effluent stream contains unsorbed components including paraffins and olefins. In the alkylation reactor, benzene is alkylated with the branched olefins in the presence of a suitable catalyst such as hydrofluoric acid or a solid, acidic catalyst. The alkylation reactor effluent stream is passed to an alkylbenzene refining section. A refined alkylbenzene product stream is obtained from the alkylbenzene refining section. The alkylbenzene product stream is withdrawn from the dehydrogenation unit 160 in line 162.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by the at least one processor, cause the one or more computing devices to perform a process that may include one or more steps. For example, the one or more computing devices may be configured to receive, from one or more monitoring components, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

The following examples are solely for purposes of illustration. These examples show in detail how the process disclosed herein claimed below may be effected and are not meant to limit the scope of the process disclosed herein to the embodiments shown in the examples. These examples are not meant to limit the scope of the process disclosed herein as set forth in the claims.

### EXAMPLES

### Example 1

Synthesis of 2,5-dihydroxymethylfuroin

In a nitrogen environment, 0.725 g of HMF was added to a vial containing 0.0612 g of TPT followed by the addition of 1.5 mL of methanol solvent. The reaction mixture was stirred at 60° C. for 1 h. As the product DHMF was formed, it precipitated out of the reaction mixture. Solvent was removed, and the product was analyzed by 1 H NMR spectroscopy to indicate 99% yield.

The procedure was repeated with different solvents. The DHMF yield was compared for different solvents and also the water content of the different solvents. Results are summarized in Table below:

TABLE

| | ppm | wt % | Mass solvent | DHMF % | mass H₂O |
|---|---|---|---|---|---|
| Solvent | H₂O | H₂O | used (g) | yield | (mg) |
| MeOH | 90 | 0.009 | 0.792 | 99.3 | 0.07128 |
| Toluene | 201 | 0.0201 | 1.7197 | 67.46 | 0.3456597 |
| Tetrahydrofuran | 250 | 0.025 | 4.51 | 45.45 | 1.1275 |
| (THF) | | | | | |

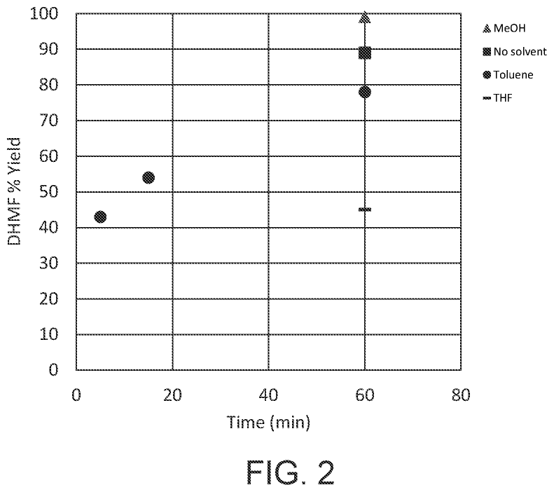

As shown in the Table, methanol produced the highest yield of DHMF. Variables such as time, temperature, feed: catalyst ratio, water content, solvent, and catalyst were investigated for different solvents and the results are shown in FIGS. 2 to 7. Operating conditions for FIGS. 2-5 were HMF: Catalyst mole ratio of 17-24; 1.5 g of solvent; time of 1 hr; strirring rotations per minute of 200; and a temperature of 60° C.

FIG. 2 is a graph plotted to show DHMF % yield vs time (min) for three different solvents: methanol, toluene, and tetrahydrofuran (THF). Also, results are shown for no solvent condition as well. As shown in FIG. 2, methanol solvent resulted in highest DHMF yield.

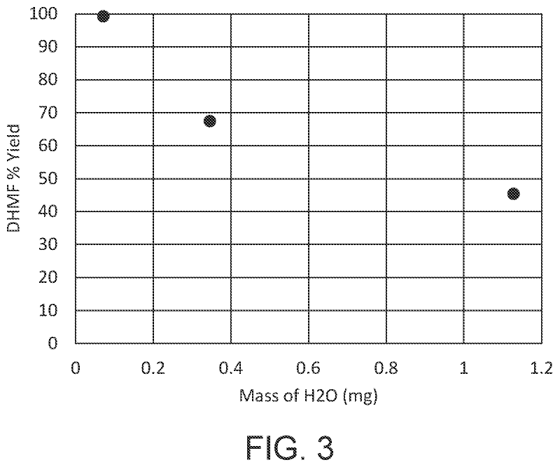

FIG. 3 is a graph plotted to show DHMF % yield vs water content of solvent.

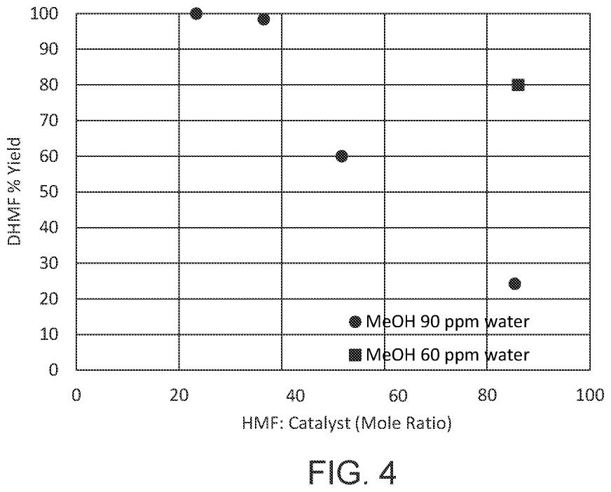

FIG. 4 is a graph plotted to show DHMF % yield vs mole ratio of HMF:catalyst to compare different water concentrations (90 ppm and 60 ppm) in methanol as the solvent.

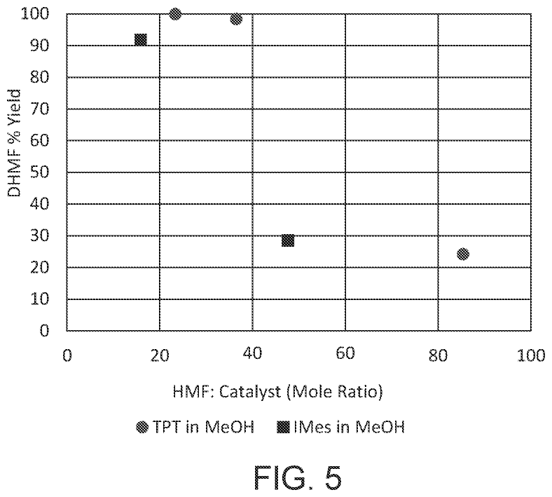

FIG. 5 is a graph plotted to show DHMF % yield vs mole ratio of HMF:catalyst for two different catalysts, TPT and 1,3-bis(2,4,6-trimethylphenyl)-1,3-dihydro-2H-imidazol-2-ylidene (IMes) in methanol as solvent. Both catalysts comprises a N-heterocyclic carbene. As shown in the FIG. 5, TPT in methanol solvent resulted in higher DHMF yield as compared to the IMes.

### Example 2

### Synthesis of Furoin

In a nitrogen environment, 0.725 g of furfural was added to a vial containing 0.0612 g of TPT followed by the addition of 1.5 ml of methanol. The reaction mixture was stirred at 60° C. for 1 h. The solvent was removed, and the product was analyzed by 1 H NMR spectroscopy. A 99% yield of the product was observed.

### Example 3

Synthesis of Dimer from Furfural and 5-Hydroxymethylfurfural

In a nitrogen environment, 0.725 g of furfural was added to a vial containing 0.0612 g of TPT followed by the addition of 1.5 ml of methanol. The reaction mixture was stirred at 60° C. for 1 h. The solvent was removed, and the product was analyzed by 1 H NMR spectroscopy to indicate 99% yield to 3 products. Three products were identified in a 1:3:1 ratio as DHMF, a heterodimer of furfural and HMF, and furoin.

### Example 4

### Reuse of NHC Catalyst

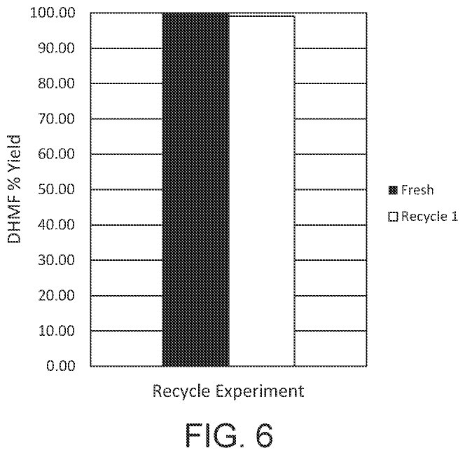

In a nitrogen environment, 0.725 g of HMF was added to a vial containing 0.0612 g of TPT followed by the addition of 1.5 mL of methanol. The reaction mixture was stirred at 60° C. for 1 h. As the product DHMF was formed, it precipitated out of the reaction mixture. After the reaction was complete, the product was collected via centrifuge. The remaining liquid contained the catalyst. Fresh HMF was added to the liquid layer that contained the catalyst, and the experimental procedure was repeated. The reaction yielded 99% DHMF according to the 1 H NMR spectroscopy. The results are shown in FIG. 6. The recycled catalyst yielded a commensurate yield of DHMF as compared to a fresh catalyst as depicted in FIG. 6.

### Example 5

### Hydrodeoxygenation of Furoin

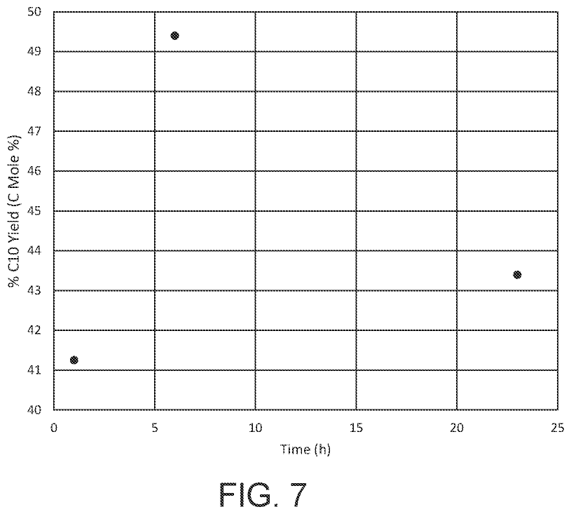

In a nitrogen environment, 1 g of furoin was loaded into a 75 mL autoclave with 7.3 g of pentadecane, and 0.3 g of pre-sulfided HYT-6319 catalyst available from UOP LLC in Des Plaines, IL. The autoclave was then filled with 500 psig to 1250 psig of hydrogen and heated to 200 to 285° C. for 1 h to 24 h. The liquid product was analyzed by GC and identified to comprise greater than 40% mol C yield of C₁₀ hydrocarbons. The results are shown in FIGS. 7 to 10.

FIG. 7 is a graph plotted to show % C10 yield (mole %) vs time (h).

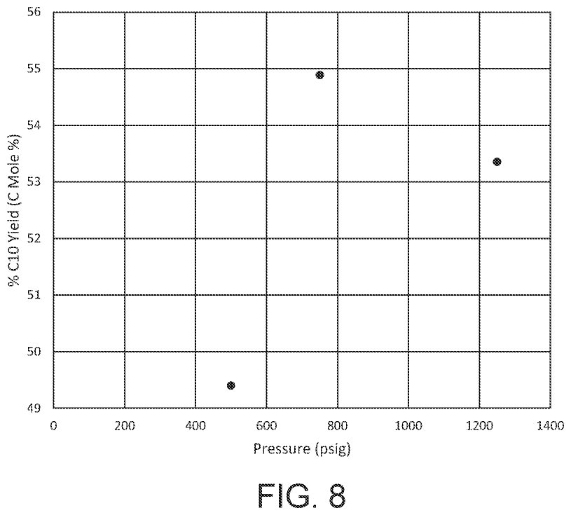

FIG. 8 is a graph plotted to show % C10 yield (mole %) vs hydrogen pressure (psig).

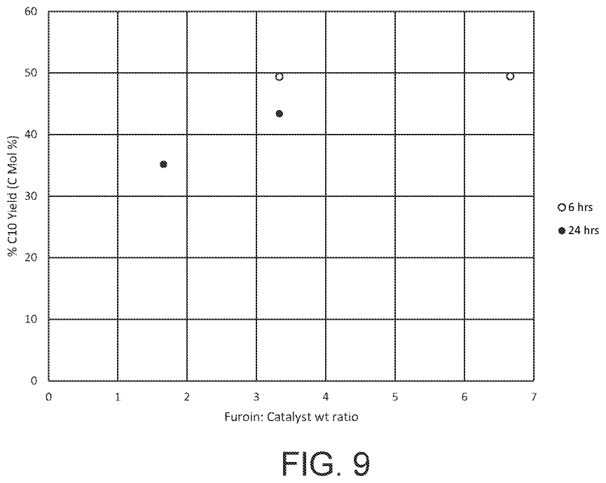

FIG. 9 is a graph plotted to show % C10 yield (mole %) vs weight ratio of furoin:catalyst for reaction time of 24 hr or 6 hr.

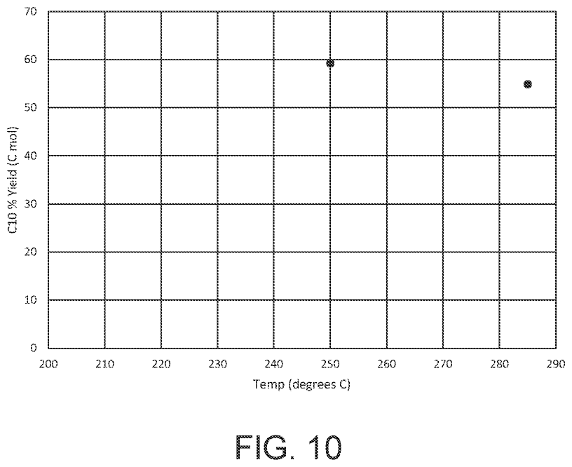

FIG. 10 is a graph plotted to show % C10 yield (mole %) vs temperature (° C.).

### Example 6

### Continuous Hydrodeoxygenation of Furoin

A 5 wt % furoin in cresol feed was passed over a catalyst of 20 cc of pre-sulfided HYT-6319 in a fixed bed at a temperature between 285 and 310° C. at 20 cc/h, 750 psig, 293 sccm H2, and a H2/feed ratio of 5000 scfb. After 15 hr on stream, n-decane was observed in a 25 mol % C yield.

### SPECIFIC EMBODIMENTS

While the following is described in conjunction with specific embodiments, it will be understood that this description is intended to illustrate and not limit the scope of the preceding description and the appended claims.

A first embodiment of the present disclosure is a process of coupling aldehydes, comprising charging an aldehyde containing stream and a catalyst stream comprising a catalyst in an alcohol solution to a reactor; contacting the aldehyde containing stream with the catalyst in the alcohol solution; precipitating a furoin based molecule in the reactor; and recovering a liquid stream comprising the catalyst in the alcohol solution from the reactor. An embodiment of the present disclosure is one, any or all of prior embodiments in this paragraph up through the first embodiment in this paragraph wherein the liquid stream comprising the catalyst in the alcohol solution is recycled to the reactor to couple a fresh supply of the aldehyde containing stream. An embodiment of the present disclosure is one, any or all of prior embodiments in this paragraph up through the first embodiment in this paragraph wherein the aldehyde containing stream comprises one or more aldehydes having formula (I)

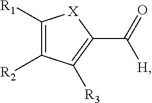

wherein R1, R2, and R3 is selected from a group comprising hydrogen, C1-C20 alkyl chains, aromatic groups, heteroatoms, and oxygenated groups comprising alcohols, ethers, carbonyls, esters, and hydroxy group, and X is selected from a group comprising heteroatoms, oxygen, sulfur, and nitrogen. An embodiment of the present disclosure is one, any or all of prior embodiments in this paragraph up through the first embodiment in this paragraph wherein the aldehyde containing stream comprises furfural. An embodiment of the present disclosure is one, any or all of prior embodiments in this paragraph up through the first embodiment in this paragraph wherein the furoin based molecule is hydrodeoxygenated to a liquid fuel. An embodiment of the present disclosure is one, any or all of prior embodiments in this paragraph up through the first embodiment in this paragraph wherein the furoin based molecule is hydrodeoxygenated and then dehydrogenated to produce a product comprising olefins having from 8 to 16 carbons. An embodiment of the present disclosure is one, any or all of prior embodiments in this paragraph up through the first embodiment in this paragraph wherein the homogeneous catalyst is a N-heterocyclic carbene catalyst. An embodiment of the present disclosure is one, any or all of prior embodiments in this paragraph up through the first embodiment in this paragraph wherein the catalyst is selected from TPT, 1,3-di-mesityl-butyl-imidazolin-2-ylidene and 1,3-dialkylimidazolin-2-ylidene. An embodiment of the present disclosure is one, any or all of prior embodiments in this paragraph up through the first embodiment in this paragraph wherein the furoin based molecule comprises one or more compound having formula (II)

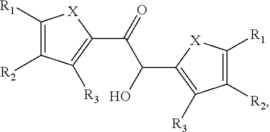

wherein R1, R2, and R3 is selected from a group comprising hydrogen, C1-C20 alkyl groups, aromatic groups, heteroatoms, and oxygenated groups comprising alcohols, ethers, carbonyls, esters, and hydroxy group, and X is selected from a group comprising heteroatoms, oxygen, sulfur, and nitrogen. An embodiment of the present disclosure is one, any or all of prior embodiments in this paragraph up through the first embodiment in this paragraph wherein furoin based molecule comprises 5,5′-di(hydroxymethyl)furoin. An embodiment of the present disclosure is one, any or all of prior embodiments in this paragraph up through the first embodiment in this paragraph wherein the reactor is a benzoin condensation reactor. An embodiment of the present disclosure is one, any or all of prior embodiments in this paragraph up through the first embodiment in this paragraph wherein the alcohol solution comprises one or more alcohols selected from methanol, ethanol, propanol, butanol, ethanolamine, phenol, and cresol.

A second embodiment of the present disclosure is a process of coupling aldehydes, comprising charging an aldehyde containing stream and a catalyst stream comprising a catalyst in an alcohol solution to a reactor; contacting the aldehyde containing stream with the catalyst in the alcohol solution; precipitating a furoin based molecule from the solution; recovering a liquid stream comprising the catalyst in the alcohol solution from the reactor; recycling the liquid stream to the reactor to couple a fresh supply of the aldehyde containing stream. An embodiment of the present disclosure is one, any or all of prior embodiments in this paragraph up through the second embodiment in this paragraph wherein the aldehyde containing stream comprises one or more aldehydes having formula (I)

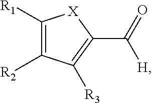

wherein R1, R2, and R3 is selected from a group comprising hydrogen, C1-C20 alkyl chains, aromatic groups, heteroatoms, and oxygenated groups comprising alcohols, ethers, carbonyls, esters, and hydroxy group, and X is selected from a group comprising heteroatoms, oxygen, sulfur, and nitrogen. An embodiment of the present disclosure is one, any or all of prior embodiments in this paragraph up through the second embodiment in this paragraph wherein the furoin based molecule is hydrodeoxygenated to a liquid fuel. An embodiment of the present disclosure is one, any or all of prior embodiments in this paragraph up through the second embodiment in this paragraph wherein the furoin based molecule is hydrodeoxygenated and then dehydrogenated to produce a product comprising a long chain linear olefin having from 8 to 16 carbons. An embodiment of the present disclosure is one, any or all of prior embodiments in this paragraph up through the second embodiment in this paragraph wherein the homogeneous catalyst is a N-heterocyclic carbene catalyst. An embodiment of the present disclosure is one, any or all of prior embodiments in this paragraph up through the second embodiment in this paragraph wherein the catalyst is selected from TPT, 1,3-di-mesityl-butyl-imidazolin-2-ylidene and 1,3-dialkylimidazolin-2-ylidene.

A third embodiment of the present disclosure is a process of coupling aldehydes, comprising charging an aldehyde containing stream and a catalyst stream comprising a catalyst in an alcohol solution to a reactor mixing the aldehyde containing stream with the catalyst in the alcohol solution and precipitating a furoin based molecule from the solution; and recovering a liquid stream comprising the catalyst in the alcohol solution from the reactor.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the present disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of coupling aldehydes, comprising:
charging an aldehyde containing stream and a catalyst stream comprising a catalyst in an alcohol solution to a reactor:
contacting said aldehyde containing stream with said catalyst in the alcohol solution;
precipitating a furoin based molecule in said reactor; and
recovering a liquid stream comprising said catalyst in the alcohol solution from the reactor.

2. The process of claim 1 wherein said liquid stream comprising said catalyst in the alcohol solution is recycled to the reactor.

3. The process of claim 1 wherein said aldehyde containing stream comprises one or more aldehydes having formula (I) 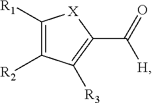
wherein
R1, R2, and R3 is selected from a group comprising hydrogen, C1-C20 alkyl chains, aromatic groups, heteroatoms, and oxygenated groups comprising alcohols, ethers, carbonyls, esters, and hydroxyls, and
X is selected from a group comprising heteroatoms, oxygen, sulfur, and nitrogen.

4. The process of claim 1 wherein the aldehyde containing stream comprises furfural.

5. The process of claim 1 wherein said furoin based molecule is hydrodeoxygenated to a liquid fuel.

6. The process of claim 1 wherein said furoin based molecule is hydrodeoxygenated and then dehydrogenated to produce a product comprising olefins having from 8 to 16 carbons.

7. The process of claim 1 wherein said homogeneous catalyst is a N-heterocyclic carbene catalyst.

8. The process of claim 7 wherein said catalyst is selected from TPT, 1,3-di-mesityl-butyl-imidazolin-2-ylidene and 1,3-dialkylimidazolin-2-ylidene.

9. The process of claim 1 wherein the furoin based molecule comprises one or more compound having formula (II) 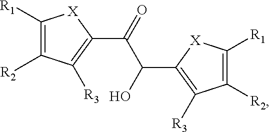
wherein
R1, R2, and R3 is selected from a group comprising hydrogen, C1-C20 alkyl groups, aromatic groups, heteroatoms, and oxygenated groups comprising alcohols, ethers, carbonyls, esters, and hydroxyls, and
X is selected from a group comprising oxygen, sulfur, and nitrogen.

10. The process of claim 1 wherein furoin based molecule comprises 5,5′-di(hydroxymethyl)furoin.

11. The process of claim 1 wherein said reactor is a benzoin condensation reactor.

12. The process of claim 1 wherein the alcohol solution comprises one or more alcohols selected from methanol, ethanol, propanol, butanol, ethanolamine, phenol, and cresol.

13. A process of coupling aldehydes, comprising:
charging an aldehyde containing stream and a catalyst stream comprising a catalyst in an alcohol solution to a reactor:
contacting said aldehyde containing stream with said catalyst in the alcohol solution;
precipitating a furoin based molecule from said solution;
recovering a liquid stream comprising said catalyst in the alcohol solution from the reactor;
recycling said liquid stream to the reactor.

14. The process of claim 13 wherein said aldehyde containing stream comprises one or more aldehydes having formula (I) 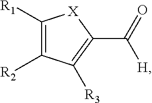
wherein
R1, R2, and R3 is selected from a group comprising hydrogen, C1-C20 alkyl chains, aromatic groups, heteroatoms, and oxygenated groups comprising alcohols, ethers, carbonyls, esters, and hydroxyls, and
X is selected from a group comprising heteroatoms, oxygen, sulfur, and nitrogen.

15. The process of claim 13 wherein the aldehyde containing stream comprises furfural.

16. The process of claim 13 wherein said furoin based molecule is hydrodeoxygenated to a liquid fuel.

17. The process of claim 13 wherein said furoin based molecule is hydrodeoxygenated and then dehydrogenated to produce a product comprising a long chain linear olefin having from 8 to 16 carbons.

18. The process of claim 13 wherein said homogeneous catalyst is a N-heterocyclic carbene catalyst.

19. The process of claim 18 wherein said catalyst is selected from TPT, 1,3-di-mesityl-butyl-imidazolin-2-ylidene and 1,3-dialkylimidazolin-2-ylidene.

20. A process of coupling aldehydes, comprising:
charging an aldehyde containing stream and a catalyst stream comprising a catalyst in an alcohol solution to a reactor:
mixing said aldehyde containing stream with said catalyst in the alcohol solution and precipitating a furoin based molecule from said solution; and
recovering a liquid stream comprising said catalyst in the alcohol solution from the reactor.
